SEQUENCE LISTING (18 sequences)

SEQ ID NO: 1 - DNA, 10 bp, Artificial Sequence
  Seed sequence
  misc_feature (8)..(10): n can be any nucleotide or absent
actcaagnnn

SEQ ID NO: 2 - DNA, 10 bp, Artificial Sequence
  Seed sequence
  misc_feature (8)..(10): n can be any nucleotide or absent
gtgagagnnn

SEQ ID NO: 3 - DNA, 21 bp, Artificial Sequence
  Mature miRNA
aactcaagcaggaaggaaggt

SEQ ID NO: 4 - DNA, 21 bp, Artificial Sequence
  Mature miRNA
tgtgagagccagctggttgtt

SEQ ID NO: 5 - DNA, 21 bp, Artificial Sequence
  Mature miRNA
aactcaagcaggaaggaggag

SEQ ID NO: 6 - DNA, 21 bp, Artificial Sequence
  Mature miRNA
tgtgagagccagctggaggag

SEQ ID NO: 7 - DNA, 59 bp, Artificial Sequence
  Pre-miRNA
aactcaagcaggaaggaaggtgttttggccactgactgacaccttcctctgcttgagtt

SEQ ID NO: 8 - DNA, 59 bp, Artificial Sequence
  Pre-miRNA
tgtgagagccagctggttgttgttttggccactgactgacaacaaccatggctctcaca

SEQ ID NO: 9 - DNA, 59 bp, Artificial Sequence
  Pre-miRNA
aactcaagcaggaaggaggaggttttggccactgactgacctcctcctctgcttgagtt

SEQ ID NO: 10 - DNA, 59 bp, Artificial Sequence
  Pre-miRNA
tgtgagagccagctggaggaggttttggccactgactgacctcctccatggctctcaca

SEQ ID NO: 11 - DNA, 10 bp, Artificial Sequence
  EXO-motif
  misc_feature (5)..(10): n can be any nucleotide or absent
ggagnnnnnn

SEQ ID NO: 12 - DNA, 10 bp, Artificial Sequence
  EXO-motif
  misc_feature (8)..(10): n can be any nucleotide or absent
ggaggagnnn

SEQ ID NO: 13 - DNA, 29 bp, Artificial Sequence
  Forward primer for HER2 PCR
cccaagcttatggagctggcggccttgtg

SEQ ID NO: 14 - DNA, 61 bp, Artificial Sequence
  Reverse primer for HER2 PCR
ataagaatgcggccgcttatcagtgatggtgatggtgatgcactggcacgtccagacccag

SEQ ID NO: 15 - DNA, 56 bp, Artificial Sequence
  miR-HER2-E1 stem-loop primer
gtcggtcgtatccagtgcagggtccgaggtattcgcactggatacgacctcctcct

SEQ ID NO: 16 - DNA, 18 bp, Artificial Sequence
  miR-HER2-E1 Forward primer
aaccaagcaggaaggagg

SEQ ID NO: 17 - DNA, 16 bp, Artificial Sequence
  miR-HER2-E1 Reverse primer
gtgcagggtccgaggt

SEQ ID NO: 18 - DNA, 14 bp, Artificial Sequence
  miR-HER2-E1 Probe
  misc_feature: 6-Carboxy-fluorescein(6-FAM) is attached to 5′ end and Minor Groove Binder(MGB) is attached to 3′ end
tcgcactggatacg

(19) United States
(12) Patent Application Publication
(10) Pub. No.: US 2023/0398144 A1
(43) Pub. Date: Dec. 14, 2023

(54) EXOSOMES CONTAINING MIRNAS TARGETING HER2 SYNTHESIS AND PHARMACEUTICAL COMPOSITIONS

(71) Applicant: IMMVIRA CO., LIMITED, Shenzhen (CN)

(72) Inventors: Xiaoqing CHEN, SHENZHEN (CN); Lei WANG, SHENZHEN (CN); Xusha ZHOU, SHENZHEN (CN); Grace Guoying ZHOU, SHENZHEN (CN)

(21) Appl. No.: 18/033,535

(22) PCT Filed: Oct. 25, 2021
(86) PCT No.: PCT/CN2021/126208

§ 371 Date: Apr. 24, 2023

(30) Foreign Application Priority Data
Oct. 26, 2020 (WO) PCT/CN2020/123619

(51) Int. Cl. A61K 35/00; C12N 15/113

(52) U.S. Cl. CPC A61K 35/00; C12N 15/1138; C12N 2310/141; C12N 2320/32

(57) ABSTRACT

Disclosed is an engineered exosome comprising a miRNA targeting human epidermal growth factor receptor 2 (HER2) synthesis and a ligand displayed on a membrane of the exosome for specific binding to a HER2 protein expressed on a cancer cell.

### TECHNICAL FIELD

The instant disclosure relates to an extracellular vesicle containing miRNAs targeting human epidermal growth factor receptor 2 (HER2) synthesis, and in particular, to an engineered exosome containing miRNAs targeting HER2 mRNA and displaying a HER2 binding ligand on the membrane of the exosome. The instant disclosure also relates to a pharmaceutical composition comprising the extracellular vesicles disclosed herein and its therapeutical use in treatment of a HER2 positive cancer.

### BACKGROUND

HER2 is a member of the human epidermal growth factor receptor family. The HER2 protein is expressed at high levels on the surface of human breast cancer cells. Its role in the oncogenic behaviors of these cells is supported by numerous observations. Indeed, treatment of HER2-positive cells with antibodies was found to block G1-S cell cycle progression, decrease the protein levels of cyclin-dependent kinase 2 (CDK2), cyclin E, and CDK6 and reduce cyclin E-CDK2-associated kinase activity. Administration of anti-HER2 antibodies is a standard of care treatment for human HER2-positive breast cancer patients.

Advances in HER2-targeted therapies have improved the survival of patients with HER2-positive breast cancer. The standard-of-care treatment for localized disease has been chemotherapy and 1 year of adjuvant HER2-targeted therapy, typically with the anti-HER2 antibody trastuzumab. Despite the effectiveness of this treatment, disease relapse occurs in a subset of patients; thus, focus has been placed on escalating treatment by either combining different HER2-targeted agents or extending the duration of HER2-targeted therapy. Indeed, dual HER2-targeted therapies and extended-duration anti-HER2 therapy, as well as adjuvant therapy with the anti-HER2 antibody-drug conjugate T-DM1, have all been approved for clinical use. Emerging evidence suggests, however, that some patients do not derive sufficient benefit from these additional therapies to offset the associated toxicities and/or costs.

### SUMMARY

An aspect of the disclosure is directed to an engineered exosome comprising a miRNA targeting human epidermal growth factor receptor 2 (HER2) synthesis. In some embodiments, the miRNA contained in the engineered exosome has a seed sequence having a nucleotide sequence as shown in SEQ ID NO. 1 or 2. The examples herein showed that a miRNA designed to target HER2 mRNA and delivered to HER2-positive cells via exosomes blocked the replenishment of HER2 and the data generated in the examples also showed that this miRNA did not affect cells that did not express HER2.

Another aspect of the disclosure is directed to an engineered exosome comprising a miRNA targeting human epidermal growth factor receptor 2 (HER2) synthesis and a ligand displayed on a membrane of the exosome for specific binding to a HER2 protein expressed on a cancer cell. The engineered exosome of this aspect achieved dual targeting of HER2 via a two-step approach. First, a HER2 binding peptide is displayed on the exosomal membrane, which enables specific entry into HER2-positive cancer cells. In the second step, a designed miRNA specifically targeting HER2 is released to downregulate HER2 protein expression. The engineered exosome of this aspect was shown herein to exhibit increased antitumor activity in vivo by systemic delivery.

A further aspect of the disclosure is directed to a method for treating a HER2-positive cancer, comprising administering to a subject in need thereof a therapeutically effective amount of any of the engineered exosomes as disclosed herein.

A yet further aspects of the disclosure is directed to a pharmaceutical composition comprising any of the engineered exosomes as disclosed herein and a pharmaceutically acceptable carrier.

Other aspects and features of the disclosure will be apparent from the detailed description that follows.

### BRIEF DESCRIPTION OF THE DRAWINGS

FIG. 1. Downregulation of HER2 by miR-HER2-E1 and miR-HER2-E4 in SK-OV-3 and HEp-2 cells. a. Immunoblotting analysis of HER2 protein levels of SK-OV-3 cells transfected with 0.5 μg of plasmids expressing miR-HER2-E1, miR-HER2-E4 or nontargeting (NT) miRNA. b. The band densities of HER2 normalized to GAPDH indicating relative HER2/GAPDH expressing in SK-OV-3 cells were quantified and are presented as the mean±standard deviation from three independent experiments. c. HEp-2 cells were co transfected with 0.5 μg of plasmid expressing miR-HER2-E1, miR-HER2-E4 or the NT miRNA and 0.2 μg of plasmid encoding His-tagged HER2. GAPDH served as a loading control. d. The band densities of HER2 normalized to GAPDH indicating relative HER2/GAPDH expressing in HEp-2 cells were quantified and are presented as the mean±standard deviation from three independent experiments. *p<0.05.

FIG. 2. Characterization of exosomes containing miR-HER2-E1. a. Immunoblotting analysis of exosomes and cells with antibodies against the exosome marker proteins Alix, CD9, Annexin V, Flotillin-1 and TSG101. b. The particle size distribution and number of isolated exosomes extracted from HEK-293 cells transfected with miR-HER2-E1 or non-targeting (NT) plasmid were measured by Izon's qNano technology (Izon). c. Quantification of miR-HER2-E1 from purified exosomes by qPCR analysis. The amount of exosomal miR-HER2-E1 was quantified and normalized to the amount of 18S rRNA. The data reported are representative of three independent experiments. All quantitative data are presented as the mean±standard deviation.

FIG. 3. Inhibition of HER2 protein accumulation by exosome-delivered miR-HER2-E1. Immunoblotting analysis of HER2 protein levels in SK-OV-3 and HEp-2 cells treated with exosome-delivered miR-HER2-E1. a. SK-OV-3 cells were left untreated (Con) or incubated with the indicated amounts of exosomes purified from HEK-293 cells transfected with miR-HER2-E1 or nontargeting (NT) miRNA. b. The band intensity quantification shown at right represents the relative HER2/GAPDH expression levels in SK-OV-3 cell. c. HEp-2 cells were transfected with the HER2 expression plasmid for 36 h and then either left untreated (Con) or incubated with 20 μg of purified exosomes produced by HEK-293 cells transfected with plasmids encoding the miR-HER2-E1 or the NT miRNAs. d. The band intensity quantification shown at right represents the relative HER2/GAPDH expression levels in HEp-2 cells. The data reported are representative of three independent experiments. All quantitative data are presented as the mean±standard deviation. *p<0.05, N.S. indicates no significant difference.

FIG. 4. miR-HER2-E1 delivered to HER2-positive cancer cells via exosomes reduces cell viability. Effects of exosome-delivered miR-HER2-E1 on the viability of HER2-positive (+) cancer cells (SK-OV-3 and HCT116) and HER2-negative (−) cells (HEp-2 and MDA-MB-231) measured by the CCK8 assay. Results are expressed as the mean of the cell viability index±standard deviation compared to the mock-treated control (as 100%). Statistically significant differences between miR-HER2-E1 exosomes and the mock-treated are indicated by an asterisk (**, p<0.01; ***, p<0.001) in the HER2-positive (+) group. N.S. indicates no significant difference.

FIG. 5. Antitumor efficacy of exosome-delivered miR-HER2-E1 in vivo. Nude mice bearing SK-OV-3 (a), HCT116 (b) or MDA-MB-231 (c) tumors (the average tumor size was 90 mm3 for each group) were injected intratumorally every three days, 6 times in total (indicated by arrow), with 10 μg of purified exosomes per injection. Tumor size was measured every three days. Results are shown as the mean tumor volume (mm3)±standard deviation (n=6). * and *** represent p<0.05 and p<0.001 compared with the NT exo group. N.S. indicates no significant difference.

FIG. 6. 293-miR-XS-HER2 expressing both a ligand for tumor cell surface HER2 and a miRNA targeting HER2. a. Accumulation of miR-HER2-E1 in stable cell lines. miR-HER2-E1 isolated from 293-miR-XS and 293-miR-XS-HER2 cell pellets and exosomes was quantified with normalization to the level of 18S rRNA. b. The relative HER2 binding affinity of 293-miR-XS and 293-miR-XS-HER2 exosomes. The absorbance readings (OD 450 nm) are shown on the Y axis. Each result is presented as the mean±standard deviation of three replicates. **p<0.01.

FIG. 7. Antitumor efficacy of exosomes adhering to HER2 and expressing miR-HER2-E1. BALB/c-derived nude mice implanted with SK-OV-3 tumors with an average volume of 80 mm3 were injected intravenously with exosomes purified from the parental HEK-293 cell line (293), the miR-HER2-E1-expressing stable cell line (293-miR-XS) or the stable cell line with coexpression of the HER2 protein ligand and miR-HER2-E1 (293-miR-XS-HER2). Exosomes were injected 3 fig/animal (a) or 30 fig/animal (b) every three days, for a total of 8 injections (indicated by arrow). The tumor size was measured every three days. The results are shown as mean tumor volume (mm3)±standard deviation (n=6). * and *** represent p<0.05 and p<0.001 compared with the 293-miR-XS group.

### DETAILED DESCRIPTION

### Definitions

It is to be noted that the term “a” or “an” entity refers to one or more of that entity; for example, “an exosome,” is understood to represent one or more exosomes. As such, the terms “a” (or “an”), “one or more,” and “at least one” can be used interchangeably herein.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art.

The term “linker” as used herein refers to a short fragment of nucleotide sequence containing two or more nucleotides which may be same or different, wherein the nucleotides are selected from a group consisting of Adenine (A), Guanine (G), Cytosine (C), Thymine (T) and Uracil (U).

As used herein, the term “treat” or “treatment” refer to both therapeutic treatment and prophylactic or preventative measures, wherein the object is to prevent or slow down (lessen) an undesired physiological change or disorder, such as the progression of tumor. Beneficial or desired clinical results include, but are not limited to, alleviation of symptoms, diminishment of extent of disease, stabilized (i.e., not worsening) state of tumor, inhibition of tumor growth, reducing the volume of the tumor, delay or slowing of tumor progression, amelioration or palliation of the tumor state, and remission (whether partial or total), whether detectable or undetectable. Those in need of treatment include those already have a tumor as well as those who are prone to have a tumor.

By “therapeutically effective amount” it is meant that the exosome of the present disclosure is administered in an amount that is sufficient for “treatment” as described above. The amount which will be therapeutically effective in the treatment of a particular individual's disorder or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on. The subject herein is preferably a human.

As used herein, phrases such as “to a patient in need of treatment” or “a subject in need of treatment” includes subjects, such as mammalian subjects, that would benefit from administration of a composition of the present disclosure used, e.g., for detection, for a diagnostic procedure and/or for treatment.

The present disclosure employs, among others, antisense oligomer and similar species for use in modulating the function or effect of nucleic acid molecules encoding HER2. The hybridization of an oligomer of this invention with its target nucleic acid is generally referred to as “antisense”. Consequently, the preferred mechanism believed to be included in the practice of some preferred embodiments of the invention is referred to herein as “antisense inhibition.” Such antisense inhibition is typically based upon hydrogen bonding-based hybridization of oligonucleotide strands or segments such that at least one strand or segment is cleaved, degraded, or otherwise rendered inoperable. In this regard, it is presently preferred to target specific nucleic acid molecules and their functions for such antisense inhibition.

The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. One preferred result of such interference with target nucleic acid function is modulation of the expression of HER2. In the context of the present disclosure, “modulation” and “modulation of expression” mean decrease (inhibition) in the amount or levels of a nucleic acid molecule encoding the gene, e.g., DNA or RNA. mRNA is often a preferred target nucleic acid.

In the context of this invention, “hybridization” means the pairing of complementary strands of oligomers. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

In the present invention the phrase “stringent hybridization conditions” or “stringent conditions” refers to conditions under which a compound of the invention will hybridize to its target sequence, but to a minimal number of other sequences. Stringent conditions are sequence-dependent and will be different in different circumstances and in the context of this disclosure, “stringent conditions” under which oligomeric compounds hybridize to a target sequence are determined by the nature and composition of the oligomers and the assays in which they are being investigated.

“Complementary,” as used herein, refers to the capacity for precise pairing between two nucleobases of an oligomeric compound. For example, if a nucleobase at a certain position of an oligonucleotide (an oligomeric compound), is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

It is understood in the art that the sequence of an antisense oligomer need not be 100% complementary to that of its target nucleic acid to be specifically hybridizable. Moreover, an oligonucleotide may hybridize over one or more segments such that intervening or adjacent segments are not involved in the hybridization event (e.g., a loop structure or hairpin structure). It is preferred that the antisense compounds of the present disclosure comprise at least 70%, or at least 75%, or at least 80%, or at least 85% sequence complementarity to a target region within the target nucleic acid, more preferably that they comprise at least 90% sequence complementarity and even more preferably comprise at least 95% or at least 99% sequence complementarity to the target region within the target nucleic acid sequence to which they are targeted. For example, an antisense compound in which 18 of 20 nucleobases of the antisense oligomer are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense oligomer which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art.

In the context of this disclosure, the term “oligonucleotide” refers to an oligomer or polymer of ribonucleic acid (RNA) or deoxyribonucleic acid (DNA) or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for a target nucleic acid and increased stability in the presence of nucleases.

As used herein, the term “microRNA”, “miRNA”, or “miR” refers to RNAs that function post-transcriptionally to regulate expression of genes, usually by binding to complementary sequences in the three prime (3′) untranslated regions (3′ UTRs) of target messenger RNA (mRNA) transcripts, usually resulting in gene silencing. miRNAs are typically small regulatory RNA molecules, for example, 21 or 22 nucleotides long. The terms “microRNA”, “miRNA”, and “miR” are used interchangeably and include both its precursor (pre-miRNA) and mature (mature miRNAs) forms. One major roadblock in functional miRNA studies is the reliable prediction of genes targeted by miRNAs, as rules defining miRNA target recognition have not been well-established to date. A seed sequence is essential for the binding of the miRNA to the mRNA. The seed sequence or seed region is a conserved heptametrical sequence which is mostly situated at positions 2-8 from the miRNA 5′-end. Even though base pairing of miRNA and its target mRNA does not match perfect, the “seed sequence” has to be perfectly complementary.

As used herein, an “antibody” or “antigen-binding polypeptide” refers to a polypeptide or a polypeptide complex that specifically recognizes and binds to one or more antigens. An antibody can be a whole antibody and any antigen binding fragment or a single chain thereof. Thus, the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. The term antibody also encompasses polypeptides or polypeptide complexes that, upon activation possess antigen-binding capabilities. Examples of these polypeptides or polypeptide complexes include Fab, Fv, scFv, and (Fab′)₂.

As used interchangeably herein, a “HER2 positive tumor” or “HER2 positive cancer” refers to a tumor or cancer that tests positive for human epidermal growth factor receptor 2 (HER2) protein which promotes the growth of cancer cells. HER2 is emerging as a promising target for genomically informed therapy across a variety of tumor types. For HER2, gene amplification (increased copy number) is by far the most common genomic alteration and is generally associated with protein overexpression. HER2 overexpression drives tumorigenesis through the creation of spontaneous receptor homodimers or heterodimers with other ERBB family members resulting in activated oncogenic downstream signaling, such as PI3K/Akt/mTOR and MAPK, promoting cellular proliferation, survival, and angiogenesis. In particular, HER2-HER3 heterodimers transduce PI3K signaling via direct binding between HER3 and the p85 subunit of PI3K. Spontaneous formation of these heterodimers increases with amplification of the HER2 gene. Algorithms for HER2 classification have been evolving. For example, for breast cancer, 3+ HER2 protein overexpression by IHC, or HER2 amplification assessed by ISH have been considered HER2-positive, and detailed guidelines for interpretation have been developed by the American Society of Clinical Oncology and College of American Pathologists and are regularly updated, which is incorporated herein by reference.

HER2-targeted therapy has transformed outcomes for HER2-amplified/overexpressing (HER2-positive) breast and gastric/gastroesophageal cancer. Several therapies are approved for HER2-positive breast cancer in the adjuvant and metastatic setting: trastuzumab (metastatic and adjuvant), pertuzumab (metastatic and adjuvant), lapatinib (metastatic), ado-trastuzumab emtansine (metastatic), and neratinib (adjuvant). Trastuzumab is also approved for HER2-positive metastatic gastric/gastroesophageal junction cancers, in combination with cisplatin and a fluoropyrimidine (capecitabine or 5-fluorouracil). Furthermore, Trastuzumab-dkst (Ogivri; Mylan), a trastuzumab biosimilar, was approved for all indications included in the label of trastuzumab.

With increased genomic profiling of many types of tumor, there is growing recognition that HER2 amplification occurs in several tumor types including salivary (3.9%), vaginal (3.6%), bladder (3.6%), endometrial (3.4%), cervical (2.2%), and colorectal cancer (1.3%).

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

Exosomes Carrying miRNA Targeting HER2

Exosomes are small extracellular vesicles averaging 50-150 nm in diameter. They serve as a means of intercellular communication. Typically, they consist of structural proteins as well as selected proteins, miRNAs, mRNAs, and long noncoding RNAs. The RNAs contain a short nucleotide sequence recognized by the proteins that transport them into the cytoplasm and package them into exosomes. Exosomes transport the payload from cell to cell. On entry into recipient cells the exosome payload is released into cytoplasm.

In the present disclosure, an engineered exosome is provided which comprises or carries miRNAs targeting mRNA of HER2 for blocking, reducing, or eliminating the replenishment of HER2 protein in a HER2 positive cancer cell.

In some embodiments, the miRNAs targeting mRNA of HER2 has a seed sequence having a nucleotide sequence of 5′-ACTCAAG-3′ (SEQ ID NO. 1), 5′-GTGAGAG-3′ (SEQ ID NO.2) or an equivalent thereof. The seed sequence is, as described above, perfectly complementary to the target mRNA of HER2. The equivalents of the seed sequences include nucleotide sequences having one or two additions or deletions of nucleotides with respect to the sequences provided herein but still exactly complementary to the target mRNA of HER2. For example, an equivalent of the seed sequence may be a hexa- or octa-metrical sequence that lacks or adds one nucleotide at the 5′- or 3′-end of the seed sequence provided herein. miRNAs targeting mRNA of HER2 may share a same seed sequence, for example that shown in SEQ ID NO. 1 of the present disclosure, but may vary largely over the whole length of the miRNAs, either precursor or mature miRNA. They are all within the scope of the present disclosure.

In preferable embodiments, the seed sequence of the miRNA targeting mRNA of HER2 is operably linked to an EXO-motif to facilitate or enhance the packaging of the miRNA into the exosome. The term “operably linked” refers to functional linkage between a regulatory sequence (e.g. the EXO-motif) and a nucleic acid sequence (e.g., the seed sequence of the miRNA) resulting in an enhance of, or facilitating the packaging of the miRNA into an exosome. For example, a first nucleic acid sequence is operably linked with a second nucleic acid sequence when the first nucleic acid sequence is placed in a functional relationship with the second nucleic acid sequence. Operably linked RNA sequences can be contiguous with each other or can be connected with a linker.

The EXO-motif is preferably located downstream the seed sequence of the miRNA, contiguously or through a linker. In one embodiment, the EXO-motif is located downstream of the seed sequence of the miRNA targeting mRNA of HER2 through a dimeric nucleotide linker. The sequences of the EXO-motif suitable for use with the present disclosure have no limit. For example, the EXO-motif suitable for use with the present disclosure has a sequence of -GGAG- (SEQ ID NO. 11), -GGAGGAG- (SEQ ID NO. 12), or an equivalent thereof. Exemplary equivalent EXO-motifs suitable for use with the present disclosure was described in WO 2020/132946 which is incorporated herein in its entirety by reference.

In addition to the seed sequence and the EXO-motif, the miRNA of the present disclosure may also include additional nucleic acid sequence to facilitate binding to the target region of the mRNA of HER2. These additional nucleic acids are normally located downstream the EXO-motif with a length of several nucleotides, e.g., 1 to 10 nucleotides. The additional nucleic acid sequences are preferably complementary to the corresponding segment of the target mRNA, but, as described above, not necessarily perfectly complementary.

Each mature miRNA is produced from a precursor transcript (pre-miRNA). After the pre-miRNA migrates from the nucleus into the cytoplasm, it is cleaved into a mature miRNA by an enzyme known as DICER. The mature miRNA molecule then binds to an RNA-induced silencing complex (RISC), which contains multiple proteins, including a ribonuclease enzyme. The miRNA nucleotide sequence directs the protein complex to bind to a complementary sequence of mRNA. Once bound to the mRNA, the miRNA-RISC complex then enzymatically cleaves targeted sites on the mRNA molecule, thereby inhibiting the translation of the gene into a protein, which effectively silences the gene.

In some embodiments, the mature form of the miRNA of the present disclosure has a nucleotide sequence as shown in any of SEQ ID NO. 3 to 6 as identified in the sequence listing provided herein, or an equivalent thereof. In some embodiments, a mature form of the miRNA of the present disclosure has a nucleotide sequence as shown in SEQ ID NOs. 5, 6 or an equivalent thereof. Mature miRNAs may include the EXO-motif as described above at the 3′-end of the sequence of the mature miRNA. In some embodiments, the EXO-motif is integrally incorporated into the sequence of the precursor or mature miRNA of the present disclosure and forms a part thereof.

In some embodiments, a precursor of the mature miRNA (pre-miRNA) of the present disclosure has a nucleotide sequence as shown in any of SEQ ID NO. 7 to 10 or an equivalent thereof. In some embodiments, a precursor of the miRNA (pre-miRNA) of the present disclosure has a nucleotide sequence as shown in SEQ ID NO. 9, 10 or an equivalent thereof. It would be appreciated that pre-miRNAs targeting HER2 may vary in length and composition of nucleotides despite they share a same seed sequence, as described above, and/or they are cleaved into a same mature miRNA. The present disclosure envisages those pre-miRNAs.

Exosomes of the present disclosure comprise pre-miRNAs, and thus mature miRNA and the seed sequences thereof, that target mRNA of HER2. Methods for transferring miRNAs into an exosome are available in the art, such as by co-transfecting a cell with a miRNA expression vector and a plasmid encoding HER2, as described in the Examples. Isolation, identification or characterization of an exosome is technically feasible in the art. Several proteins, e.g. CD9, CD63, CD81, CD82, Annexin, Flotillin, etc can be used as a marker of exosomes. Other methods for packaging miRNAs into exosomes may also be applicable with the present invention.

### HER2 Dual-Targeting Exosomes

A further aspect of the present disclosure relates to a HER2 dual-targeting exosome which carries miRNA targeting HER2 mRNA within the exosome and displays a ligand on the exosomal membrane targeting HER2 protein on a cancer cell surface. The HER2 dual-targeting exosome of the present disclosure directs the engineered exosome near or close to a HER2 positive cancer cell where HER2 protein is expressed through the binding of the ligand displayed on the exosomal membrane to the HER2 protein, and then enters into the HER2 positive cancer cell and releases the miRNA cargo to downregulate the expression of the HER2 protein within the cancer cell.

The miRNA targeting HER2 mRNA as carried by the HER2 dual-targeting exosome of the present disclosure is as set forth above with reference to the exosomes carrying miRNA targeting HER2. Based on that, the exosome is further engineered to display a ligand for binding to a HER2 protein expressed on a HER2 positive cancer cell.

In some embodiments, the ligand is a peptide having an affinity for HER2, preferably with specificity to HER2 protein. Suitable ligands for the present disclosure may be, for example, a fragment of an anti-HER2 antibody, for example, the antigen-binding fragment of an anti-HER2 monoclonal antibody. In some embodiments, the fragment of an anti-HER2 antibody is selected from a group consisting of Fab, Fv, scFv and F(ab′)₂. Suitable anti-HER2 antibodies are those approved by FDAs, i.e. trastuzumab and pertuzumab. Moreover, fragments of investigational anti-HER2 monoclonal antibodies are expected to be used with the present disclosure, e.g. Margetuximab by MacroGenics, Inc.

Methods for displaying a peptide ligand on the membrane of an exosome is already available in the market, for example, the XStamp™ Technology developed by System Biosciences (SBI).

### Methods and Therapies

A further aspect of the disclosure provides a method for treatment of cancer in a subject in need thereof comprising administering to the subject a therapeutically effective amount of the engineered exosome described herein.

In some embodiments, in the methods of the disclosure, the administering of the exosome is carried out by administering to the subject a pharmaceutical composition comprising the exosome.

In certain embodiments, any of the pharmaceutical composition is administered parenterally or non-parenterally, e.g. intratumorally, intravenously, intramuscularly, percutaneously or intracutaneously. In some embodiments, any of the pharmaceutical compositions is preferably administered intratumorally. In some embodiments, any of the pharmaceutical compositions is preferably administered intravenously.

The methods of the disclosure are contemplated to treat various HER2 positive cancers, including cancers with HER2 amplification and/or overexpression. In some embodiments, the exosome described herein is used to treat a cancer that is dependent on HER2 for its survival. Examples of HER2 positive tumors that can be treated according to the disclosure include but are not limited to breast, gastric, salivary, vaginal, bladder, endometrial, cervical and colorectal cancers.

### Pharmaceutical Compositions

A further aspect of the disclosure provides a pharmaceutical composition comprising any of the exosomes as provided herein and a pharmaceutically acceptable carrier. The pharmaceutical composition is useful for prophylaxis or treatment of a HER2 positive cancer in a subject.

Under ordinary conditions of storage and use, these preparations/compositions contain a preservative to prevent the growth of microorganisms. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, sodium chloride or phosphate buffered saline. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion.

Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDAs.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

### SEQUENCE LISTING

The following a summary of sequences as mentioned in the present disclosure. An electronic form of the sequences is prepared and affixed herein.

| SEQ | | |
|---|---|---|
| ID | Names | Sequences |
| 1 | Seed sequence | 5′-ACTCAAG-3′ |
| 2 | Seed sequence | 5′-GTGAGAG-3′ |
| 3 | Mature miRNA | 5′-AACTCAAGCAGGAAGGAAGGT-3′ |
| 4 | Mature miRNA | 5′-TGTGAGAGCCAGCTGGTTGTT-3′ |
| 5 | Mature miRNA | 5′-AACTCAAGCAGGAAGGAGGAG-3′ |
| 6 | Mature miRNA | 5′-TGTGAGAGCCAGCTGGAGGAG-3′ |
| 7 | Pre-miRNA | 5′-AACTCAAGCAGGAAGGAAGGTGTT |
| | | TTGGCCACTGACTGACACCTTCCTCT |
| | | GCTTGAGTT-3′ |
| 8 | Pre-miRNA | 5′-TGTGAGAGCCAGCTGGTTGTTGT |
| | | TTTGGCCACTGACTGACAACAACCAT |
| | | GGCTCTCACA-3′ |
| 9 | Pre-miRNA | 5′-AACTCAAGCAGGAAGGAGGAGGT |
| | | TTTGGCCACTGACTGACCTCCTCCTC |
| | | TGCTTGAGTT-3′ |
| 10 | Pre-miRNA | 5′-TGTGAGAGCCAGCTGGAGGAGGT |
| | | TTTGGCCACTGACTGACCTCCTCCAT |
| | | GGCTCTCACA-3′ |
| 11 | EXO-motif | 5′-GGAG-3′ |
| 12 | EXO-motif | 5′-GGAGGAG-3′ |
| 13 | Forward primer | 5′-CCCAAGCTTATGGAGCTGGCGG |
| | for HER2 PCR | CCTTGTG-3′ |
| 14 | Reverse primer | 5′-ATAAGAATGCGGCCGCTTATCAG |
| | for HER2 PCR | TGATGGTGATGGTGATGCACTGGCAC |
| | | GTCCAGACCCAG-3′ |
| 15 | miR-HER2-E1 | 5′-GTCGGTCGTATCCAGTGCAGGGT |
| | stem-loop | CCGAGGTATTCGCACTGGATACGACC |
| | primer | TCCTCCT-3′ |
| 16 | miR-HER2-E1 | 5′-AACCAAGCAGGAAGGAGG-3′ |
| | Forward primer | |
| 17 | miR-HER2-E1 | 5′-GTGCAGGGTCCGAGGT-3′ |
| | Reverse primer | |
| 18 | miR-HER2-E1 | 5′-(6-FAM) TCGCACTGGATACG |
| | Probe | (MGB)-3′ |

### Examples

### Methods and Materials

Purchased cell lines: The HEp-2 cell line (human laryngeal carcinoma cells) was purchased from the American Type Culture Collection (ATCC). The HEK-293 cell line (human embryonic kidney 293 cells) and SK-OV-3 cell line (human ovarian epithelial cancer cells) were purchased from the Cell Bank of the representative culture preservation committee of the Chinese Academy of Sciences (Shanghai, China). The HCT116 cell line (human colorectal carcinoma cells) and the MDA-MB-231 cell line (human breast cancer cells) were kindly provided by Professor Jun Du (Sun Yat-sen University, Guangzhou, China). HEp-2 cells were cultured in DMEM (high-glucose) supplemented with 5% (vol/vol) fetal bovine serum (FBS). HEK-293 and MDA-MB-231 cells were maintained in DMEM (high-glucose) containing 10% (vol/vol) FBS. SK-OV-3 cells were cultured in McCoy's 5A medium supplemented with 10% (vol/vol) FBS. All culture media contained 100 U/ml penicillin and 100 μg/ml streptomycin. Cells were incubated in a humidified atmosphere containing 5% CO₂ at 37° C.

Generation of stable cell lines: The stable cell line 293-miR-HER2, expressing miRNA targeting HER2, was generated by transfection of the miR-HER2-E1 plasmid into HEK-293 cells. Forty-eight hours after transfection, cells were selected by the addition of blasticidin (Solarbio Life Sciences) to a final concentration of 6 μg/ml. A cell colony with green fluorescent protein (GFP) expression was selected and cultured in complete medium with 6 μg/ml blasticidin. The cell line was monitored for the expression of GFP and miR-HER2-E1.

To generate a cell line producing exosomes that adhere to the surface of HER2-positive cells, 293-miR-HER2 cells were either infected with lentivector XSTP724PA-1 (XStamp HER2 ligand exosome HER2 receptor targeting lentivector) or infected with control lentivector XSTP710PA-1 according to the manufacturer's instructions (XStamp Technology, SBI: XSTP724PA-1/XSTP710PA-1). The two cell lines were renamed 293-miR-XS-HER2 and 293-miR-XS (control), respectively. The lentivector XSTP724PA-1 contains two copies of the HER2 ligand fused to the 5′ N-terminal signal sequence leader and fused in frame to the 3′ C-terminal C1C2 XStamp domain that directs the entire fusion protein to be displayed on the surface of secreted exosomes. The HER2 binding ability of the exosomes purified from 293-miR-XS-HER2 cells was confirmed by ELISA.

Antibodies: The anti-HER2 (Cat. No. #21655) antibody was purchased from Cell Signaling Technology. The anti-GAPDH, anti-Alix, anti-CD9, anti-Annexin V, anti-Flotillin-1, and anti-TSG101 antibodies have been described elsewhere.

Plasmid construction: The miRNA sequences targeting the HER2 gene were designed using BLOCK-iT™ RNAi Designer (Life Technologies) and synthesized by Ige Biotechnology (Guangzhou, China). The synthesized miRNA fragments were digested with the BamHI and XhoI restriction enzymes and cloned into the corresponding sites in the pcDNA6.2-GW/EmGFP-miR-neg control plasmid (Invitrogen). The miRNA sequences were as follows:

miR-HER2-1:
(SEQ ID NO. 7)
5′-AACTCAAGCAGGAAGGAAGGTGTTTTGGCCAC
TGACTGACACCTTCCTCTGCTTGAGTT-3′
miR-HER2-4:
(SEQ ID NO. 8)
5′-TGTGAGAGCCAGCTGGTTGTTGTTTTGGCCAC
TGACTGACAACAACCATGGCTCTCACA-3′
miR-HER2-E1:
(SEQ ID NO. 9)
5′-AACTCAAGCAGGAAGGAGGAGGTTTTGGCCAC
TGACTGACCTCCTCCTCTGCTTGAGTT-3′
miR-HER2-E4:
(SEQ ID NO. 10)
5′-TGTGAGAGCCAGCTGGAGGAGGTTTTGGCCACT
GACTGACCTCCTCCATGGCTCTCACA-3′

The sequences of the mature miRNAs are underlined. miR-HER2-E1 and miR-HER2-E4 were modified versions of miR-HER2-1 and miR-HER2-4, respectively, as they contained EXO-motifs.

The HER2 expression plasmid containing the 3′-UTR sequence of the HER2 gene was generated as follows. First, the His-tagged HER2 coding sequence was amplified by PCR using the following primers:

Forward,
(SEQ ID NO. 13)
5′-CCCAAGCTTATGGAGCTGGCGGCCTTGTG-3′
and
Reverse,
(SEQ ID NO. 14)
5′-ATAAGAATGCGGCCGCTTATCAGTGATGGTGA
TGGTGATGCACTGGCACGTCCAGACCCAG.

The PCR fragment was then subcloned into pcDNA3.1(+) at the HindIII/NotI sites to generate pHER2-His. The 3′-UTR sequence was synthesized by Ige Biotechnology (Guangzhou, China), digested with the NotI and XbaI restriction enzymes and cloned into the corresponding sites in pHER2-His.

Exosome isolation and quantification: HEK-293 cells (1×10 7) seeded in a T150 flask were mock transfected or transfected with 10 μg of the NT or miR-HER2-E1 plasmid. After 4 h of incubation, the cells were rinsed extensively with phosphate-buffered saline (PBS) and incubated in serum-free medium for an additional 48 h. Cells of the stable cell lines were seeded in a T150 flask for 24 h, rinsed extensively with PBS and incubated in serum-free medium for another 48 h. Cell-free extracellular medium containing exosomes was harvested by centrifugation at 300×g for 10 min to remove the cells. The supernatant was then centrifuged at 10,000×g for 30 min to remove dead cells and cell debris. Finally, the clear supernatant was centrifuged for 70 min at 100,000×g to pellet the exosomes. All centrifugation steps were carried out at 4° C. For immunoblotting, the pelleted exosomes were resuspended in RIPA buffer. For treatment of cells or mice, the pelleted exosomes were resuspended in PBS. The exosomes were quantified by the BCA method using an Enhanced BCA Protein Assay Kit (Beyotime) according to the manufacturer's instructions.

Exosome size analysis: The size distribution of the exosomes was analyzed using the Izon qNano system (Izon, Christchurch, New Zealand; www.izon.com), which uses single-molecule electrophoresis to detect extracellular vesicles passing through a nanopore. The procedure yielded accurate particle-by-particle enumeration of exosomes ranging from 75 to 300 nm in diameter. Specifically, purified exosomes were diluted 1:10 in PBS containing 0.05% Tween 20, shaken vigorously, and measured by using an NP200 (A53942) nanopore aperture according to the manufacturer's instructions. The results were analyzed using Izon Control Suite software v3.3 (Izon Science).

Quantitative RT-PCR for miRNA: Total RNA from cells and suspended exosomes was isolated using TRIzol reagent (Thermo Fisher Scientific) and TRIzol LS reagent (Thermo Fisher Scientific) according to the manufacturer's instructions. The miRNAs tested were reverse transcribed from 50 ng of total RNA in duplicate with specific stem-loop primers as described in the TaqMan miRNA reverse transcription kit instructions (Applied Biosystems, Inc.). miRNA expression was measured by real-time PCR using a TaqMan Universal Master Mix II kit purchased from Applied Biosystems, Inc. The miRNA copy number was normalized to that of cellular 18S rRNA. The primers specific for miR-HER2-E1 were designed and then synthesized by Ige Biotechnology. The sequences follow:

miR-HER2-E1 stem-loop primer,
(SEQ ID NO. 15)
5′-GTCGGTCGTATCCAGTGCAGGGTCCGAGGTA
TTCGCACTGGATACGACCTCCTCCT-3′;
Forward primer,
(SEQ ID NO. 16)
5′-AACCAAGCAGGAAGGAGG-3′;
Reverse primer,
(SEQ ID NO. 17)
5′-GTGCAGGGTCCGAGGT-3′;
Probe,
(SEQ ID NO. 18)
5′-(6-FAM) TCGCACTGGATACG (MGB)-3′.

Plasmid transfection: SK-OV-3 or HEp-2 cells were seeded in 12-well plates at 2.5×10 5 cells per well. Cells were transfected with 0.5 μg of plasmids expressing miR-HER2-E1, miR-HER2-E4 or non-targeting (NT) miRNA or for HEp-2 cells were contransfected with 0.2 μg of a plasmid encoding His-tagged HER2 using Lipofectamine 2000 reagent (Thermo Fisher Scientific) following manufacturer's instructions. Cells were harvested at 72 h after transfection and used for immunoblotting analysis.

Exosomes incubation: SK-OV-3 cells at 2.5×10 5 cells per well were exposed to different concentrations of purified exosomes (0.1 μg, 5 μg, or 20 μg) carrying miR-HER2-E1 or purified exosomes (20 μg) produced by NT-transfected HEK-293 cells or remain no treatment (Con) for 72 h. For HEp-2 cells at a density of 2.5×10 5 per well were transfected with 0.2 μg of a plasmid encoding His-tagged HER2 for 36 h and then incubated 20 jig of purified exosomes produced by HEK-293 cells transfected with plasmids encoding the miR-HER2-E1 or the NT miRNAs or remain no treatment for another 36 h. The cells were then harvested for endogenous and exogenous HER2 expression by immunoblotting analysis.

Immunoblotting assay: Cell pellets or purified exosomes were harvested and lysed with RIPA lysis buffer (Beyotime) supplemented with the protease inhibitor phenylmethylsulfonyl fluoride (PMSF) (1 mM; Beyotime) and a phosphatase inhibitor (Beyotime). Cell lysates and exosomes were heat denatured at 100° C. incubator for 10 min, separated by 10% or 8% SDS-PAGE and transferred to polyvinylidene difluoride membranes (Millipore). The proteins were identified by incubation with the appropriate primary antibody and then with an HRP-conjugated secondary antibody (Pierce). Immunoreactions were visualized with ECL reagent (Pierce), and images were acquired using a ChemiDoc Touch Imaging System (Bio-Rad) and analyzed with Image Lab software. The densities of the corresponding bands were quantified using ImageJ software.

Cell viability assay: SK-OV-3, HCT116, HEp-2 and MDA-MB-231 cells were seeded into 96-well plates at a density of 1×10⁴ cells per well one day before exposure to exosomes. Cells in triplicate wells were mock treated (Mock) or incubated with 1 jig of purified exosomes produced by HEK-293 cells transfected with plasmids encoding either miR-HER2-E1 or the nontargeting (NT) miRNA. After 72 h of incubation, the relative cell viability was determined by a CCK8 assay according to the manufacturer's protocol.

Animal models: BALB/c nude mice at 6-7 weeks of age were purchased from Vital River Laboratory Animal Technologies Co., Ltd. (Beijing, China). The nude mice were injected subcutaneously in the flanks with 5×10⁶ SK-OV-3, HCT116 or MDA-MB-231 cells respectively. In the exosome-delivered miR-HER2-E1 treatment, mice with tumors having an average volume of 90 mm3 were injected intratumorally with 50 μl containing 10 μg of purified exosomes per injection. Each tumor-bearing animal was injected on days 1, 4, 7, 10, 13, and 16, for a total of 6 injections. The size of tumors was measured on days 1, 4, 7, 10, 13, 16, 19 and 22. In the exosome-delivered 293-miR-XS-HER2 treatment nude mice derived from BALB/c were injected subcutaneously into flanks with 5×10 6 SK-OV-3 cells. Tumors averaging 80 mm³ were injected intravenously on days 1, 4, 7, 10, 13, 16, 19 and 22 with 3 μg/animal or 30 μg/animal of exosomes purified from 293-miR-XS-HER2, 293-miR-XS or parental HEK-293 cells. The sizes of tumors were measured on days 1, 4, 7, 10, 13, 16, 19, 22, 25 and 28 using a caliper, and the volume was calculated as (length×width 2)×0.5.

ELISA: Exosomes purified from the 293-miR-XS or 293-miR-XS-HER2 stable cell lines were coated (1 μg/well) in triplicate wells of 96-well ELISA plates (Corning). After the coating solution was removed, nonspecific binding sites were blocked by incubation with 2% BSA at 37° C. for 1 h. The plates were rinsed, exposed to HER2 protein (Sino Biological, China) for 2 h, rinsed again, and reacted with the HRP-conjugated rabbit anti-HER2 antibody (Cat. No. 1004-R205, Sino Biological) for an additional 1 h. The plates were then rinsed again and exposed to TMB for color development. The reaction was terminated by the addition of stop solution. The plates were read in a BioTek microplate reader at a wavelength of 450 nm.

Design and Identification of miRNAs with EXO-Motifs Capable of Suppressing HER2 Synthesis

The objective of the first series of experiments was to design miRNAs targeting HER2. To this end, we constructed 7 miRNAs and selected the miRNA that most effectively in blocked HER2 synthesis in a HER2-positive cancer cell line and in a cell line transfected with a plasmid encoding HER2. The miRNA sequences were cloned into a miRNA expression vector named pcDNA6.2-GW/EmGFP-miR-neg downstream of an open reading frame encoding EGFP, as described in the Materials and Methods.

In the initial screening, SK-OV-3, a cell line with high HER2 expression, and HEp-2, a HER2-negative cell line transfected with a plasmid encoding HER2, were transfected with the plasmids encoding the miRNAs. Of the 7 miRNAs tested, miRNAs No. 1 and No. 4 most effectively suppressed the accumulation of HER2 on protein level. On the basis of these results, miRNAs No. 1 and No. 4 were selected for further studies and designated as miR-HER2-1 and miR-HER2-4, respectively.

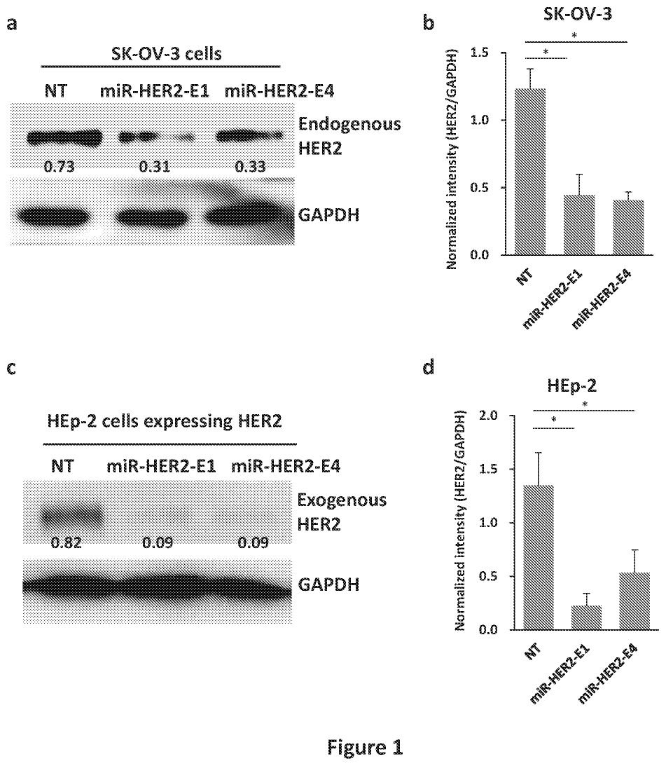

Next, we modified these two miRNAs by the addition of sequences containing exosome-packaging-associated motifs (EXO-motifs). The HER2 suppression efficacy of miR-HER2-E1 and miR-HER2-E4 was then retested in SK-OV-3 cells and in HEp-2 cells expressing HER2. The protein accumulation analyzed by immunoblotting showed that both miR-HER2-E1 and miR-HER2-E4 can significantly down-regulate the endogenous HER2 expression in SK-OV-3 cells (FIG. 1a, b) as well as exogenous HER2 expression in HEp-2 cells transfected with HER2 plasmid (FIG. 1c, d). On the basis of these results, we selected miR-HER2-E1 for further studies. The sequences of miR-HER2-1, miR-HER2-4, miR-HER2-E1 and miR-HER2-E4 are listed in the Materials and Methods.

Production and Characterization of Exosomes-Encapsulated miR-HER2-E1

In all experiments described in this disclosure, we used exosomes produced by HEK-293 cells. The properties of these exosomes were investigated as described in brief below.

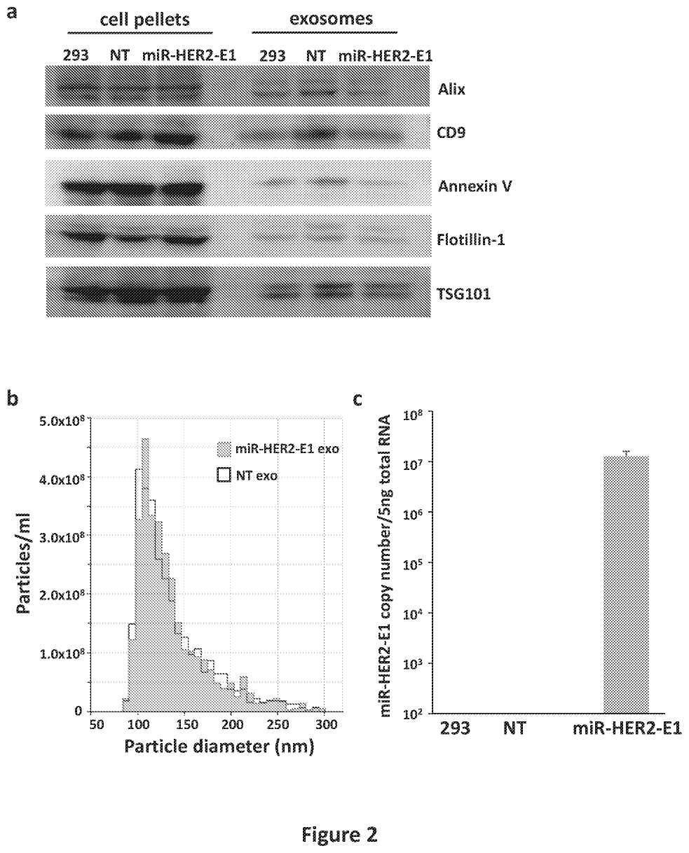

To assess the protein content of exosomes, purified exosomes derived from HEK-293 cells untreated (293) or transfected with non-targeting (NT) or miR-HER2-E1 plasmids were analyzed via Immunoblotting. Alix, CD9, Annexin V, Flotillin-1, and TSG101 were used as marker proteins of exosomes. As expected, the exosome-associated proteins were present in purified exosomes derived from untreated HEK-293, NT miRNA-transfected and miR-HER2-E1-transfected cells (FIG. 2a).

To determine the size distribution of exosomes, exosomes purified from HEK-293 cells transfected with miR-HER2-E1 were analyzed by Izon's qNano technology as described in the Materials and Methods. The results (FIG. 2b) showed that exosomes from HEK-293 cells has similar size distributions as those cells transfected with NT or miR-HER2-E1, with diameters ranging from 75-150 nm.

FIG. 2c shows the expression level of mature miR-HER2-E1 as measured by quantitative PCR (qPCR). As expected, high level of mature miR-HER2-E1 was detected in exosomes derived from HEK-293 cells transfected with miR-HER2-E1 plasmid, which indicated that Exo-motifs containing in miR-HER2-E1 contribute to package miRNA into exosomes. By contrast, the exosomes purified from parental HEK-293 cells, and the NT miRNA-transfected cells did not contain detectable amounts of miR-HER2-E1.

Exosome-Delivered miR-HER2-E1 Decreases the Accumulation of HER2 and Reduces the Viability of Cells with High Levels of HER2 Expression

In this series of experiments, we examined whether miR-HER2-E1 produced in HEK-293 cells and delivered via exosomes effectively blocked the accumulation of HER2. We report 2 series of experiments below.

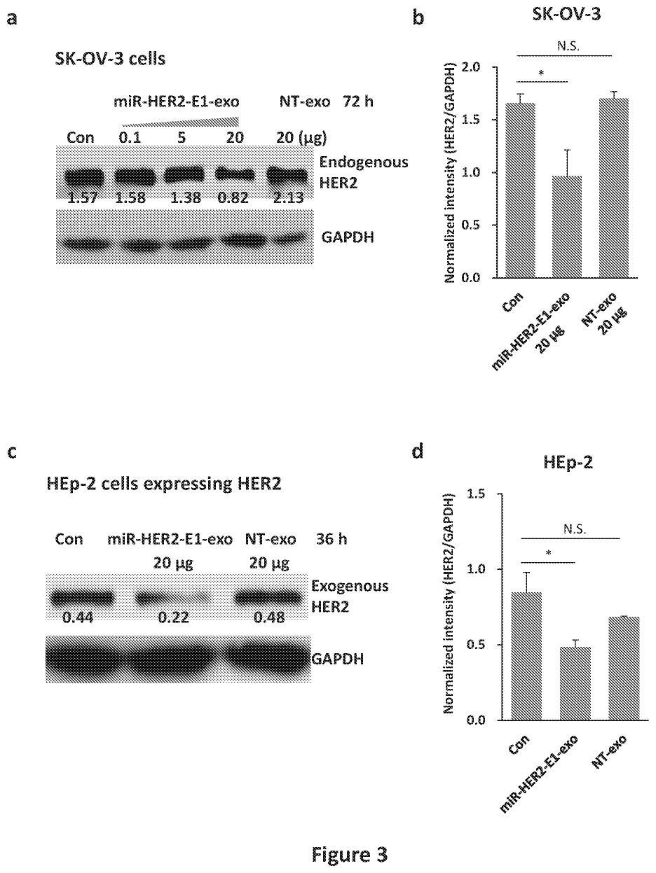

In the first series of experiments, the SK-OV-3 cells was exposed to different concentrations of purified exosomes carrying miR-HER2-E1 or purified exosomes produced by NT-transfected HEK-293 cells or remain no treatment (Con). It shows that the accumulation of HER2 decreased dose-dependently in SK-OV-3 cells exposed to exosomes containing miR-HER2-E1. Especially, HER2 expression in SK-OV-3 cells decreased significantly when treated with the highest concentration (20 μg) of purified exosomes-encapsulated miR-HER2-E1, while exosomes derived from NT transfected cells showed no effect on HER2 expression (FIG. 3a, b).

The second series of experiments was designed to determine whether miR-HER2-E1-expressing exosomes could also suppress the accumulation of exogenous HER2. HEp-2 cells were transfected with a plasmid encoding HER2 and were then exposed to purified exosomes mentioned as above. FIGS. 3c and d showed that the expression of exogenous HER2 was decreased in cells exposed to exosomes containing miR-HER2-E1 but not in exosomes derived from NT-transfected HEK-293 cells.

Downregulation of HER2 Expression Via Exosome-Delivered miR-HER2-E1 can Inhibit the Viability of HER2-Positive Cancer Cell

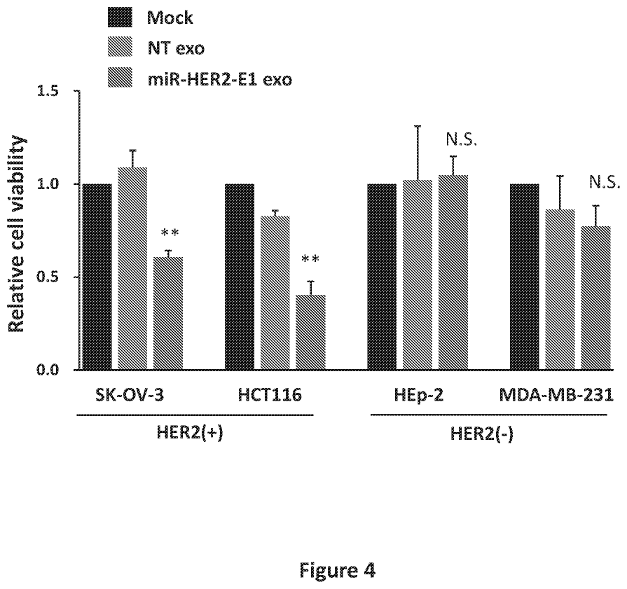

To test the hypothesis that exosome-delivered miR-HER2-E1 has tumoricidal effects on HER2-dependent cells by blocking replenishment of the protein in HER2-positive SK-OV-3 and HCT116 cancer cells as well as in HER2-negative MDA-MB-231 and HEp-2 cancer cells, the four cell lines were exposed to 1 μg of exosomes purified from HEK-293 cells transfected with miR-HER2-E1 respectively. And the relative cell viability was determined by a CCK8 assay. The results (FIG. 4) showed the following:
(i) SK-OV-3 cells exposed to exosomes carrying miR-HER2-E1 exhibited a viability of 60% relative to that of SK-OV-3 cells treated with exosomes carrying NT miRNA;
(ii) HCT116 cells exposed to exosomes carrying miR-HER2-E1 exhibited a viability of 40% compared to the 90% viability of HCT116 cells treated with exosomes carrying NT miRNA;
(iii) HEp-2 and MDA-MB-231 cells, the two types of HER2-negative cells, exhibited a viability of 90-100% upon exposure to exosomes carrying miR-HER2-E1.

These results suggest the following: (i) miR-HER2-E1 delivered by exosomes has anti-tumor effects on HER2-dependent cells by blocking the replenishment of the protein, and (ii) miR-HER2-E1 does not affect the viability of cells that are not dependent on HER2 for survival.

Antitumor Efficacy of miR-HER2-E1 Delivered by Administration of Exosomes In Vivo

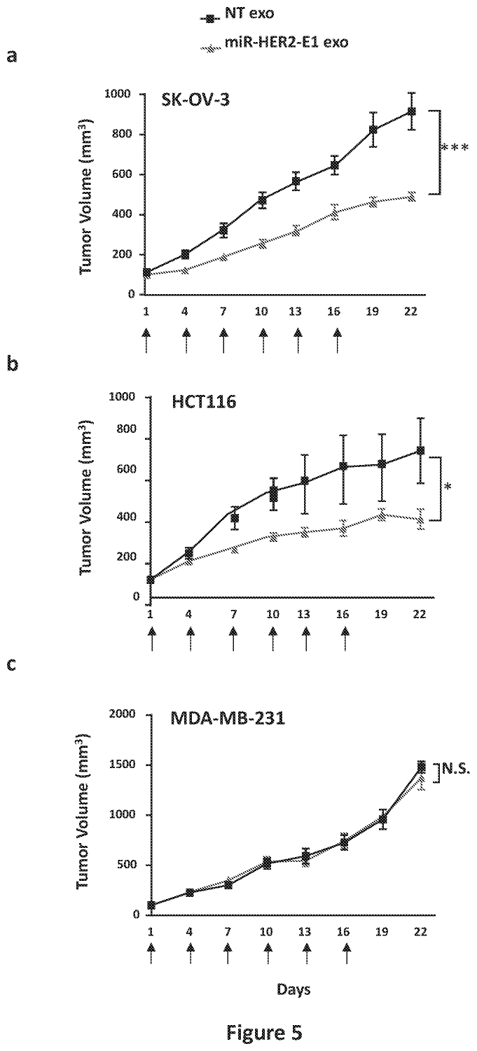

The SK-OV-3 (HER2-positive), HCT116 (HER2-positive) or MDA-MB-231 (HER2-negative) cells were used as tumor model for the in vivo study to evaluate the antitumor efficacy of miR-HER2-E1 delivered by exosomes via intratumoral administration. Ten micrograms of exosomes purified from plasmid transfected HEK-293 cells were injected intratumorally every three days for a total 6 injections. The size of the tumors was measured every three days. The results (FIG. 5) showed that exosomes carrying miR-HER2-E1 caused a reduction in the volume of SK-OV-3 tumors (FIG. 5a) and HCT116 tumors (FIG. 5b) but not tumors induced by HER2-negative MDA-MB-231 tumor cells (FIG. 5c). These results indicated that miR-HER2-E1 delivered by exosomes can interfere HER2 expression and specifically inhibit HER2-positive tumor growth in vivo, which were consistent with the results obtained in cell culture studies.

### Generation of Stable Cell Line Producing HER2 Dual-Targeting Exosomes

The results of the experiments described above in the text showed that exosomes carrying miR-HER2-E1 entered both HER2-positive and HER2-negative cells but ultimately displayed tumoricidal effects only on HER2-positive cells. Tumors are heterogeneous and are likely to contain both HER2-positive cancer cells as well as HER2-negative cells. Although our studies indicated that exosomes carrying miRNAs targeting HER2 did not affect HER2-negative cells, it was nevertheless desirable to increase the uptake of exosomes carrying HER2-targeting miRNAs by HER2-positive tumor cells. To this end, we modified the exosomes with a surface peptide by which they bound to HER2 on the surface of cancer cells. The objective of the studies described below was to enhance the entry efficiency of exosomes into HER2-positive cells. To achieve this objective, we generated two cell lines designed to produce novel exosomes. The surface of 293-miR-XS-HER2 exosomes carried a peptide that enabled these exosomes to adhere to HER2 on the surface of HER2-positive cells. In contrast, the surface of 293-miR-XS exosomes lacked the HER2 adhesion peptide. The miR-HER2-E1 was packaged into both types of exosomes. Details of the construction and characterization of the two generated cell lines are described in the Materials and Methods.

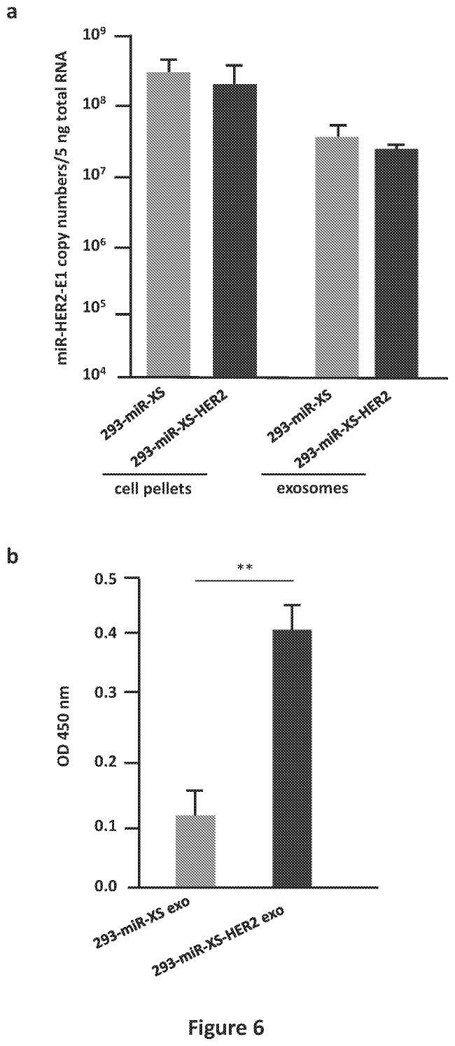

Firstly, we verified the presence of miR-HER2-E1 in exosomes produced by both cell lines. As shown in FIG. 6a, the exosomes produced by both cell lines contained mature miR-HER2-E1, as determined by analysis of cell pellets and purified exosomes.

Next, the exosomes produced by the two cell lines were purified and tested by ELISA to verify their ability to adhere to HER2 protein. As shown in FIG. 6b, purified exosomes produced by 293-miR-XS-HER2 cells preferentially bound HER2 protein. The results indicate that the dual-targeting 293-miR-XS-HER2 exosomes package miR-HER2-E1 targeting HER2 gene and display a HER2-directed peptide on their surface, which enable 293-miR-XS-HER2 exosomes preferentially deliver HER2 miRNA into HER2 positive cells.

Antitumor Efficacy of Exosomes Carrying miR-HER2-E1 and Adhering to HER2

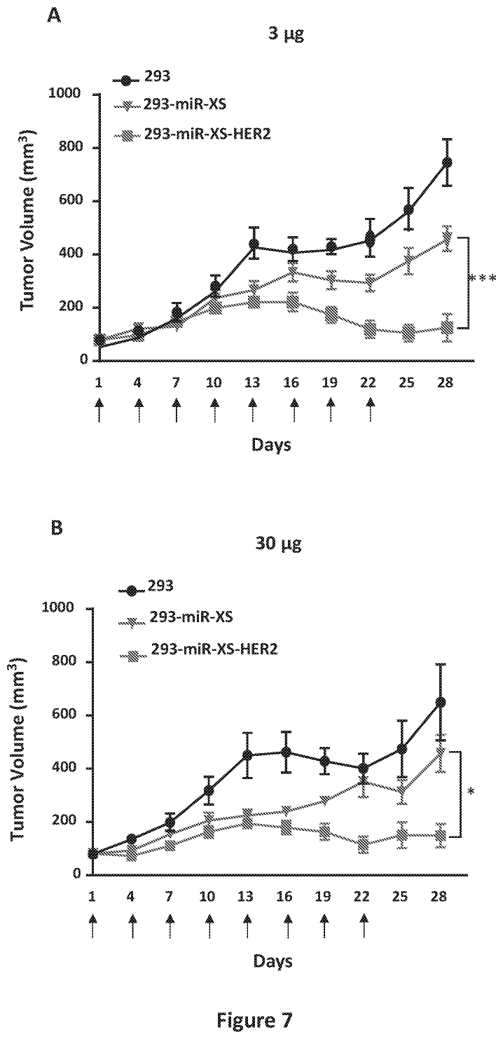

To verify whether the HER2-dual targeting exosomes (293-miR-XS-HER2) have improved antitumor efficacy compared with HER2 single targeting miRNA only (293-miR-XS) and non-targeting exosomes (293) in vivo by intravenous administration, HER2-positive tumor cells SK-OV-3 were transplanted into BALB/c nude mice. The results showed that compared to exosomes purified from HEK-293 or 293-miR-XS cells, exosomes purified from 293-miR-XS-HER2 cells were significantly more effective in reducing the growth of HER2-positive tumors (FIGS. 7a and b). Moreover, the reductions in the sizes of tumors injected with 3 μg and 30 μg of 293-miR-XS-HER2 exosomes/mouse were virtually identical, suggesting that the 3 μg dose was close to or greater than the dose required to show tumoricidal effects on susceptible cells. Consequently, we significantly enhanced the uptake of exosomes carrying miRNAs directed against HER2 by HER2-positive cells.

It should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

All publications, patent applications, patents, and other references mentioned herein are expressly incorporated by reference in their entirety, to the same extent as if each were incorporated by reference individually. In case of conflict, the present specification, including definitions, will control. The disclosures illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

1. An engineered exosome comprising a miRNA targeting human epidermal growth factor receptor 2 (HER2) synthesis and a ligand displayed on a membrane of the exosome for specific binding to a HER2 protein expressed on a cancer cell.

2. The engineered exosome of claim 1, wherein the miRNA has a seed sequence having a nucleotide sequence as shown in SEQ ID NO. 1, 2 or an equivalent thereof.

3. The engineered exosome of claim 2, wherein the seed sequence of the miRNA is operably linked to an EXO-motif.

4. The engineered exosome of claim 3, wherein the EXO-motif is located downstream of the seed sequence.

5. The engineered exosome of claim 3, wherein the EXO-motif has a sequence of -GGAG-, -GGAGGAG- or an equivalent thereof.

6. The engineered exosome of claim 1, wherein a mature form of the miRNA has a nucleotide sequence as shown in any of SEQ ID NO. 3 to 6 or an equivalent thereof.

7. The engineered exosome of claim 1, wherein a mature form of the miRNA has a nucleotide sequence as shown in SEQ ID NO. 5, 6 or an equivalent thereof.

8. The engineered exosome of claim 1, wherein a precursor of the miRNA has a nucleotide sequence as shown in any of SEQ ID NO. 7 to 10 or an equivalent thereof.

9. The engineered exosome of claim 1, wherein a precursor of the miRNA has a nucleotide sequence as shown in SEQ ID NO. 9, 10 or an equivalent thereof.

10. The engineered exosome of claim 1, wherein the ligand is a peptide having an affinity for the HER2 protein expressed on the surface of the cancer cell.

11. The engineered exosome of claim 1, wherein the ligand is a peptide specifically binding to the HER2 protein expressed on the surface of the cancer cell.

12. The engineered exosome of claim 1, wherein the ligand is a fragment of an anti-HER2 antibody.

13. The engineered exosome of claim 12, wherein the fragment of an anti-HER2 antibody is selected from a group consisting of Fab, Fv, scFv and F(ab′)2.

14. The engineered exosome of claim 1, wherein the cancer cell is dependent on HER2 for its survival.

15. The engineered exosome of claim 1, wherein the cancer cell has an overexpression or amplification of HER2.

16. The engineered exosome of any of claim 1, wherein the cancer cell is selected from a group consisting of breast, gastric, salivary, vaginal, bladder, endometrial, cervical and colorectal cancer cells.

17. An engineered exosome comprising a miRNA targeting human epidermal growth factor receptor 2 (HER2) synthesis, wherein the miRNA has a seed sequence having a nucleotide sequence as shown in SEQ ID NO. 1, 2 or an equivalent thereof.

18. The engineered exosome of claim 17, wherein the seed sequence of the miRNA is operably linked to an EXO-motif.

19. The engineered exosome of claim 18, wherein the EXO-motif is located downstream of the seed sequence.

20. The engineered exosome of claim 18, wherein the EXO-motif has a sequence of -GGAG-, -GGAGGAG- or an equivalent thereof.

21. The engineered exosome of claim 17, wherein a mature form of the miRNA has a nucleotide sequence as shown in any of SEQ ID NO. 3 to 6 or an equivalent thereof.

22. The engineered exosome of claim 17, wherein a mature form of the miRNA has a nucleotide sequence as shown in SEQ ID NO. 5, 6 or an equivalent thereof.

23. The engineered exosome of claim 17, wherein a precursor of the miRNA has a nucleotide sequence as shown in any of SEQ ID NO. 7 to 10 or an equivalent thereof.

24. The engineered exosome of claim 17, wherein a precursor of the miRNA has a nucleotide sequence as shown in SEQ ID NO. 9, 10 or an equivalent thereof.

25. The engineered exosome of claim 17, wherein the exosome is originated from HEK-293 cell.

26. A pharmaceutical composition comprising the engineered exosome of claim 1, and a pharmaceutically acceptable carrier.

27. The pharmaceutical composition of claim 26, wherein the pharmaceutical composition is formulated in injectable form.

28. The pharmaceutical composition of claim 26, wherein the pharmaceutical composition is formulated for intravenous administration.
